SEQUENCE LISTING (36 sequences)

SEQ ID NO: 1 - DNA, 1970 bp, Homo sapiens
  5′UTR (1)..(71)
  3′UTR (1951)..(1970)
atattggagcagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgagggaaaccctcgctgggagcaaacacatctgaggtacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaagttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctagataggcaagggataactcttctaacacaaaataagtgttttatgtttggaataaagtcaaccttgtttctactgtttt

SEQ ID NO: 2 - DNA, 25 bp, Homo sapiens
ggagcagcaagaggctgggaagcca

SEQ ID NO: 3 - DNA, 25 bp, Homo sapiens
aagaggctgggaagccatcacttac

SEQ ID NO: 4 - DNA, 25 bp, Homo sapiens
actgagaaagaagacaaaggccagt

SEQ ID NO: 5 - DNA, 25 bp, Homo sapiens
caaaggccagtatgcacagctttcc

SEQ ID NO: 6 - DNA, 25 bp, Homo sapiens
atgcacagctttcctccactgctgc

SEQ ID NO: 7 - DNA, 25 bp, Homo sapiens
gtgacagtaagctaacctttgatgc

SEQ ID NO: 8 - DNA, 25 bp, Homo sapiens
aagggaataagtactgggctgttca

SEQ ID NO: 9 - DNA, 25 bp, Homo sapiens
atccaagttatcccaaaatgatagc

SEQ ID NO: 10 - DNA, 25 bp, Homo sapiens
gaaggtgttttcctgaagaactgtc

SEQ ID NO: 11 - DNA, 25 bp, Homo sapiens
ctcagtcatttttaacctctagagt

SEQ ID NO: 12 - DNA, 25 bp, Homo sapiens
cactgatacacagaatataatctta

SEQ ID NO: 13 - DNA, 25 bp, Homo sapiens
ggcttatggattcatataggccaga

SEQ ID NO: 14 - DNA, 920 bp, Homo sapiens
  5′UTR (1)..(39)
  3′UTR (839)..(920)
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgt

SEQ ID NO: 15 - DNA, 15 bp, Homo sapiens
ataaacattctctgt

SEQ ID NO: 16 - DNA, 25 bp, Homo sapiens
ccccagccccaccatgaccctcggc

SEQ ID NO: 17 - DNA, 25 bp, Homo sapiens
ccagccccaccatgaccctcggccg

SEQ ID NO: 18 - DNA, 25 bp, Homo sapiens
actcgcgtgtcttttcctcgcctgt

SEQ ID NO: 19 - DNA, 25 bp, Homo sapiens
ccatgggctggggccttctgggcag

SEQ ID NO: 20 - DNA, 25 bp, Homo sapiens
actccggcagccccttggtctgcaa

SEQ ID NO: 21 - DNA, 25 bp, Homo sapiens
gatgcctttgccccggtggcacagt

SEQ ID NO: 22 - DNA, 25 bp, Homo sapiens
aactggatcgactctatcatccaac

SEQ ID NO: 23 - DNA, 25 bp, Homo sapiens
atctggcacaataaacattctctgt

SEQ ID NO: 24 - DNA, 2582 bp, Mus musculus
  5′UTR (1)..(308)
gggcagtttttttgtcttaactgactatgtgtgactaaaaggccgagttttacctgcaacccagcgtctgagaatatggccccatccatcccttcatagaagagtagtggtcttaaggaaataatagtattaaaatgtttatttctatgcccaagaaaagacattagcaaaatctacagtcaacaacagatgattggcagcacctctatttcctgtgtcatgatattcccgtgggaacccttctgtgttttggaaacaatcaaataaacctttgagcacacttccggcatgctgaaaacagcaatccatgtttatccagtgggtgacacagcttggtgaactggtgctcttcgtccttctggtcgctccggcagctctgaaacccgcaatgcctcctgtgatcaaggaccatcctttcaacgttttctgggccgcccctactttgttttgtaaagataatttcaatgtaaacatgaatcttcaagtatttaacatcattccgaatcctttcgaaactcagagtggatccactattaccgtattttatccaaaggaactcgggtattacccttatttctctgaagatggaacatccttctatggtggaatacctcagaaagtgaacctctctgagcacctcaggaaaagtgccggcgacattgcagatgctgtcactttgtggagatcggaaggacttgctgttatagactgggaaggctggaggccccagtgggataggaattggggcagtagaatgatatataaaaaccactcgctagccttcactcgacaccaccatcctgactgggcagaaacaaaagtgagaacagctgcccagaaagaatttgaaaatgctggaagaagttttatgaatgttactctcacgttggctttagaaatgagaccaaagcgtttatggggcttctatctctatccagattgctacaattatgattaccggataaatccagagttctacacaggaagttgcccagatgatgaaattttccgcaatgaccaactcatgtggctatgggaaaaaagtgcagcactttatccttccatatatttgagtaaaatattaaaatcaaacttaaatgcattgaaatttgttcatttccgagtgagagaagccctgagggttgccgaaatggctagaaaggactatgttttgccagttttcattttttccagaccattttatttgcaaagtattgaagctctctcagaggaggatttagtgcacacaattggcgaaagtgctgcattgggagcagcaggaataatattgtggggaggatatgaatattcggatacaaaggagacttgcttatctgtgagacaaactgttcatgggctattgggtccctatgttctcaatgtaacatcggcagcaaagctctgtagtcaaaatctatgtaacagtcatggaagatgtgtacggaagacacctgaatcttccttttatctgcatatgcctgaggatagccataagaattatgtgtcaaagaagggtttcagatttgtcattccctccccaagtaagctgaagacaataatgaatatgaagaatggatttgtgtgccactgttactatggctggcatggagactcctgcagatctcactctccaaatctccaaaagaacaaagctcctgcaagtggattgaattcagcagttattgttggcatggccttatttgtgattctgatgaattattttcctattccctactacaatggcaatttttccttgaaaccactgaagagaagagaaattatctttctttgatttcaagcattttctaaagaatttttagttttaagtttcatacaaattgttggatgttcacttcatgctgggcaataggaatagacctgtcctcgtggatcttagaaattaagaataaataagcagtaaatagacacatatgatagtgacaatggtttgtagaataaagagaaaacaggtttaaggcattaggaagtactagagagcactggaaattgtataaaagattgtcacataaggcctttatagtgagaaaatcagataacagatgaattaggtcaacactatcagggagcgagatattagtttcctcctataatctgagcacctggaaggcagagatccattacaaattcaaagccagcttggcttacaaactgagctaaagtcagtcaagacaacatagtgaaagttttgtcttcaaaaaaggaaaatagaagcaaaatagcaaagatccaaaagaacgaatctggggaaagagttcttgaaataaaaaggagcaaacccagaagagacaactctgcatttcagggcttgtaaacttttaaataacaagcaacacaacaatagcaaaacaacagcagcagcagcagcaacaatgatactcaccgatggagatgaaaatgactcttccaatggtttcatcaatgattagctttatccagcggacatttggttcatcaataccaaaagagaactctaccagcttcctacacaataggatttatgtcagaaattcttttgtttgatactctttggtttattgacatatactatgcccaaacagtcg

SEQ ID NO: 25 - DNA, 25 bp, Homo sapiens
ggcagcacctctatttcctgtgtca

SEQ ID NO: 26 - DNA, 24 bp, Homo sapiens
tcccgtgggaacccttctgtgttt

SEQ ID NO: 27 - DNA, 25 bp, Homo sapiens
aacctttgagcacacttccggcatg

SEQ ID NO: 28 - DNA, 25 bp, Homo sapiens
ctgaaaacagcaatccatgtttatc

SEQ ID NO: 29 - DNA, 25 bp, Homo sapiens
tttatccagtgggtgacacagcttg

SEQ ID NO: 30 - DNA, 25 bp, Homo sapiens
tgcctgaggatagccataagaatta

SEQ ID NO: 31 - DNA, 25 bp, Homo sapiens
gttggcatggccttatttgtgattc

SEQ ID NO: 32 - DNA, 25 bp, Homo sapiens
gaaaccactgaagagaagagaaatt

SEQ ID NO: 33 - DNA, 25 bp, Homo sapiens
aaattgttggatgttcacttcatgc

SEQ ID NO: 34 - DNA, 25 bp, Homo sapiens
ggcaataggaatagacctgtcctcg

SEQ ID NO: 35 - DNA, 25 bp, Homo sapiens
agtgacaatggtttgtagaataaag

SEQ ID NO: 36 - DNA, 25 bp, Homo sapiens
aagattgtcacataaggcctttata

(19) United States
(12) Patent Application Publication
(10) Pub. No.: US 2006/0025363 A1
(43) Pub. Date: Feb. 2, 2006

(54) Use of antisense oligonucleotides for the treatment of degenerative skin conditions

(76) Inventors: Ute Breitenbach, Hamburg (DE); Stephan Gallinat, Wedel (DE); Ludger Kolbe, Dohren (DE); Claudia Mundt, Bremen (DE); Volker Schreiner, Hamburg (DE); Franz Stab, Echem (DE); Rainer Wolber, Hamburg (DE); Helga Biergiesser, Reinbek (DE); Heiko Mielke, New Wulmstorf (DE); Thomas Blatt, Wedel (DE); Kirsten Venzke, Emden (DE); Kyra Sanger, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 11/064,037

(22) Filed: Feb. 22, 2005

Related U.S. Application Data
(63) Continuation of application No. PCT/EP03/09178, filed on Aug. 18, 2003.

(30) Foreign Application Priority Data
Aug. 21, 2002 (DE) 102 38 298.0

(51) Int. Cl. A61K 48/00; C12Q 1/68

(52) U.S. Cl. 514/44; 435/6

(57) ABSTRACT

A pharmaceutical or cosmetic composition for topical application containing one or more oligonucleotides which are capable of hybridizing with a mRNA or a gene sequence which codes for a connective-tissue decomposing enzyme, or a physiologically compatible salt thereof. The compositions are suitable in particular for the treatment of degenerative skin conditions.

The invention relates to compositions containing antisense oligonucleotides which are suitable for the treatment and prophylaxis of degenerative skin conditions, in particular those associated with skin aging.

Chronological skin aging is caused by endogenous, genetically determined factors and manifests itself in age-related structural damage and dysfunctions in the epidermis and dermis of the skin, such as dryness, roughness and development of dry lines/wrinkles, itching and reduced rehydration by sebaceous glands (e.g. after washing). These symptoms are collectively called “senile xerosis”.

Endogenous aging processes can be accelerated and aggravated by exogenous factors such as UV light and chemical noxa. In addition, exogenous influences can cause further structural damage and dysfunctions in the epidermis and dermis of the skin, such as for example visible vascular dilatations (telangiectasis, cuperosis), slackness and formation of wrinkles, local hyper-, hypo- and mispigmentations (e.g. age marks) and increased susceptibility to mechanical stress (e.g. tendency to crack).

Skin aging and wrinkling as a consequence of UV exposure are accompanied by a reduction in skin elasticity and by changes in elastic fibres in the dermis. Histological and ultrastructural studies showed that the biggest changes in skin that had been aged by UV radiation manifest themselves in the connective tissue (Scharffetter-Kochanek K, Wlaschnek M, Brenneisen P, Schauen M, Blaudschun R, Wenk J. UV-induced reactive oxygen species in photocarcinogenesis and photoaging. Biol. Chem. 1997 November; 378 (11): 1247-57).

Here, the structural damage and dysfunctions caused by exogenous and endogenous factors are called degenerative skin conditions.

Known products for the care of aged skin can contain, in addition to rehydrating constituents, e.g. retinoids (vitamin A acid and/or its derivatives) or vitamin A and/or its derivatives. Tsukahara, K., Y. Takema, et al. describe for example the use of retinoic acid to reduce wrinkling. This is said to effect a regeneration of the elastic fibres (Tsukahara, K., Y. Takema, et al. (2001). “Selective inhibition of skin fibroblast elastase elicits a concentration-dependent prevention of ultraviolet B-induced wrinkle formation.” J Invest Dermatol 117 (3): 671-7).

Active ingredients such as retinol can trigger complex metabolic processes in the cell, vitamin A generally being an initiator for cell regeneration. The substance detaches dead corneocyte cells, replenishes wrinkles from the inside and improves the skin structure.

The effect of these products on structural damage is limited, however. In addition, vitamin A acid-containing products can cause pronounced erythematous skin irritations. Retinoids can therefore be used only in low concentrations. Moreover, there are considerable difficulties during product development in stabilizing the active ingredients sufficiently against oxidative decomposition.

Nor does the use of agents for protection against UV radiation provide extensive protection against degenerative skin changes.

In the literature, the use of tetracyclines and batimastat to inhibit metalloproteinases (MMPs) in cancers is also described. Metalloproteinases play an important part in the decomposition of the connective tissue, in particular the collagen fibres.

Mehta et al., The Journal of Investigative Dermatology 115 (2000) 805, describe the treatment of psoriasis by inhibiting the expression of the intercellular adhesion molecule 1 (ICAM-1), the expression of which is increased in psoriasis plaques and which is therefore regarded as an attractive target for psoriasis treatment with phosphothioate antisense oligonucleotides.

The use of antisense oligonucleotides for the treatment of degenerative skin conditions has not been described to date.

The object of the present invention is the provision of compositions which make possible an effective treatment of degenerative skin states and in particular skin states due to aging, without displaying the disadvantages of the state of the art.

This object is achieved by pharmaceutical or cosmetic compositions for topical application which contain one or more oligonucleotides.

Compositions are preferred which contain oligonucleotides which are capable of hybridizing with a mRNA or a gene sequence which code for an enzyme which decomposes connective tissue. The oligonucleotides according to the invention are accordingly antisense oligonucleotides. By enzymes which decomposes connective tissue is meant primarily peptidases, in particular endopeptidases, and glycosaminoglycan-decomposing enzymes, in particular hyaluronic acid-decomposing endo-N-acetylglucosamimidases, in particular hyaluronidases. Hyaluronic acid is also called hyaluronane.

In addition to the named oligonucleotides, physiologically compatible salts of such oligonucleotides are also suitable according to the invention. For simplicity's sake, the term oligonucleotide will be used hereafter for both the actual oligonucleotides and for their salts, unless otherwise stated. The term oligonucleotide also includes modified forms of DNA and RNA.

Preferred endopeptidases include primarily collagen-decomposing and elastin-decomposing endopeptidases, in particular matrix metalloproteinases (MMPs) and elastases. Preferred MMPs include the following enzymes which can be divided into collagenases and non-collagenases:

| | |
|---|---|
| MMP-1 | P03956 (EC 3.4.24.7) |
| MMP-2 | P08253 (EC 3.4.24.24) |
| MMP-3 | P08254 (EC 3.4.24.17) |
| MMP-7 | P09237 (EC 3.4.24.23) |
| MMP-8 | P22894 (EC 3.4.24.34) |
| MMP-9 | P14780 (EC 3.4.24.35) |
| MMP-10 | P09238 (EC 3.4.24.22) |
| MMP-11 | P24347 (EC 3.4.24) |
| MMP-12 | P39900 (EC 3.4.24.65) |
| MMP-13 | P45452 (EC 3.4.24) |
| MMP-14 | P50281 (EC 3.4.24) |
| MMP-15 | P51511 (EC 3.4.24) |
| MMP-16 | P51512 (EC 3.4.24) |
| MMP-17 | Q9ULZ9 (EC 3.4.24) |
| MMP-19 | Q99542 (EC 3.4.24) |
| MMP-20 | 060882 (EC 3.4.24) |
| MMP-24 | Q9Y5R2 (EC 3.4.24) |
| MMP-25 | Q9NPA2 (EC 3.4.24) |
| MMP-26 | Q9NRE1 (EC 3.4.24) |
| MMP-28 | Q9H239 (EC 3.4.24) |

The enzymes MMP 1, 8 and 13 are collagenases, the other named enzymes non-collagenases. The numbers given are the accession numbers of the Swiss PROT EMBL-EBI database (European Bioinformatics Institute Heidelberg).

Preferred elastases include the enzymes which are isolated from the pancreas, from macrophages and from leukocytes, in particular the enzyme ELA2 (P08246 EC 3.4.21.37).

Preferred endo-N-acetylglucosaminidases include:

| | |
|---|---|
| SPAM1 | (s67798) |
| HYAL3 | (AF036035) |
| HYAL4 | (AF009010) |
| HYAL5 | (AF036144) |

and in particular HYAL2 (AK016575). The accession numbers given here are those of the NCBI database (National Center for Biotechnology Information) of the National Institute of Health.

In each case oligonucleotides are preferred which are complementary to the 3′ or 5′ untranslated region, the open reading frame, the translation-initiating region or the region adjacent to the start sequence of the genes or the corresponding regions of the mRNA of the genes of the named enzymes.

Compositions which contain oligonucleotides which are capable of hybridizing with the genes or mRNAs of collagen-decomposing endopeptidases are particularly preferred. Collagen-decomposing endopeptidases (collagenases) are enzymes which degrade the structure proteins of the connective tissue and are responsible for the decomposition of elastin and collagen fibres, but also of proteoglycans. The controlled activity of these enzymes plays a decisive role in tissue restructuring during development, tissue repair and angiogenesis processes.

Compositions are quite particularly preferred which contain oligonucleotides which can hybridize with the mRNA or the gene of zinc-dependent endopeptidases (matrix metalloproteinases, MMPs), in particular the matrix metalloproteinases 1, 8 and 13, quite particularly preferred the matrix metalloproteinase 1. These enzymes are described e.g. in Fisher G J, Choi H C, Bata-Csorgo Z, Shao Y, Datta S, Wang Z Q, Kang S, Voorhees J J., Ultraviolet irradiation increases matrix metalloproteinase-8 protein in human skin in vivo, J Invest Dermatol. 2001 August; 117(2):219-26. These are antisense oligonucleotides which can hybridize with sequence sections of SEQ ID NO 1. SEQ ID NO 1 is the cDNA of this enzyme.

Oligonucleotides which can hybridize with the gene or the mRNA of the matrix metalloproteinase 9 are equally preferred. It is assumed that this, together with the metalloproteinases 1, 8 and 13, is involved in the process caused by UV radiation, of the so-called “photoaging” of the skin.

Of the oligonucleotides which can hybridize with the SEQ ID NO 1, those are particularly preferred which can hybridize with the untranslated region downstream from nucleotide 1951 onwards (3′ untranslated region), the region from nucleotide 72 to 1481 (open reading frame), the translation initiating region (region from nucleotide 72 to 369), the region upstream from nucleotide 71 onwards (5′ untranslated region) and quite particularly preferably with the region adjacent to the start sequence (72-128).

Oligonucleotides which can hybridize with one of the sequences SEQ ID NO 2 to SEQ ID NO 13 are further preferred.

Moreover, compositions which contain oligonucleotides which are capable of hybridizing with the genes or mRNAs of elastases, preferably serin proteinases, such as pancreatic and neutrophilic elastases and macrophage elastase are particularly preferred according to the invention.

From the mechanistic point of view, elastases (pancreatic and neutrophilic elastases, macrophage-elastase) play an important role in the degeneration of elastic fibres. These serin proteinases participate among other things in phagocytotic processes, in defence against microorganisms, the degradation of elastin, collagens, proteoglycans, fibrinogen and fibrin and the digestion of damaged tissue (Bolognesi, M., K. Djinovic-Carugo, et al. (1994). “Molecular bases for human leucocyte elastase inhibition.” Monaldi Arch Chest Dis 49 (2): 144-9).

In particular, neutrophilic elastase is accorded great significance in the development of solar elastosis (Starcher, B. and M. Conrad (1995). “A role for neutrophil elastase in solar elastosis.” Ciba Found Symp 192: 338-46; discussion 346-7). Biochemical studies have shown that human dermal fibroblasts from skin with dermal elastosis have high levels of elastase and cathepsin G (Fimiani, M., C. Mazzatenta, et al. (1995). “Mid-dermal elastolysis: an ultrastructural and biochemical study.” Arch Dermatol Res 287 (2): 152-7).

Oligonucleotides are particularly preferred which can hybridize with the SEQ ID NO 14, in particular those which can hybridize with the untranslated region downstream from nucleotide 839 onwards (3′ untranslated region), the region from nucleotide 39 to 842 (open reading frame), the translation initiating region (nucleotide 39-119), the region upstream from nucleotide 39 onwards (5′ untranslated region) and quite particularly the region adjacent to the start sequence (nucleotide 39-75). Oligonucleotides which can hybridize with SEQ ID NO 15 to SEQ ID NO 23 are further preferred. The cDNA of the elastase ELA2 has the SEQ ID NO 14.

Compositions also particularly preferred according to the invention are those which contain oligonucleotides which are capable of hybridizing with the genes or mRNAs of hyaluronidases, preferably the already named enzymes SPAM1 (s67798), HYAL3 (AF036035), HYAL4 (AF009010), HYAL5 (AF036144) and particularly preferably HYAL2 (AK016575).

Oligonucleotides are quite particularly preferred which can hybridize with the SEQ ID NO 24, in particular those which can hybridize with the region from nucleotide 308 to 1792 (open reading frame), the translation initiating region (308-498), the region upstream from nucleotide 308 onwards (5′ untranslated region) and quite particularly the region adjacent to the start sequence (308-421). Further preferred are oligonucleotides which can hybridize with SEQ ID NO 25 to SEQ ID NO 36. The cDNA of hyaluronidase 2 has the SEQ ID NO 24.

The compositions according to the invention can contain one or preferably more oligonucleotides. These can be nucleotides which can hybridize with the gene sequences or mRNAs of several different collagen-decomposing enzymes, elastases and/or hyaluronidases and/or with different sequence regions of one and the same gene or the same mRNA of a collagen-decomposing enzyme, an elastase or a hyaluronidase.

In the case of collagen-decomposing enzymes, oligonucleotides which are directed against one or more of the sequences SEQ ID NO 1 to SEQ ID NO 13 are preferred, in the case of elastases, oligonucleotides which are directed against one or more of the sequences SEQ ID NO 14 to SEQ ID NO 23, and in the case of hyaluronidases, oligonucleotides which are directed against one or more of the sequences SEQ ID NO 24 to SEQ ID NO 36.

Compositions are quite particularly preferred which in each case contain at least one oligonucleotide which is directed against a collagen-decomposing enzyme, an elastase and a hyaluronidase.

Oligonucleotides are particularly suitable which at 27 to 47° C., preferably at 27 to 37° C. and quite particularly preferably at 32° C., at a pH value of 4 to 9, preferably 5 to 8, and at physiological osmolarity, salt and electrolyte concentration hybridize specifically with the named genes or gene sections or their mRNAs.

The oligonucleotides according to the invention are preferably 7 to 50 nucleotides long, particularly preferably 9 to 35 nucleotides, quite particularly preferably 12 to 30 nucleotides. Very good results were also achieved with oligonucleotides with 15 to 27, in particular 20 to 26 or exactly 25 nucleotides. They preferably display, relative to 10 bases, a maximum of 0 to 4, particularly preferably 0 to 2 and quite particularly preferably no mismatches.

It surprisingly transpired that the antisense oligonucleotides contained in the compositions according to the invention, following application to the skin, hybridize with the genes or mRNAs which code for the enzymes essential for the decomposition of the connective tissue and thus by intervention modulate the expression of the enzymes essential for the connective-tissue-decomposition process, i.e. directly and specifically inhibit the transcription and/or translation of these enzymes, in particular also of alternative splice forms, and thus prevent the degeneration of collagen, elastin and/or hyaluronic acid without side-effects and in this way make possible an effective treatment and prophylaxis of degenerative skin conditions without displaying the disadvantages of the state of the art.

The oligonucleotides according to the invention can be in the form of oligoribo- or oligodeoxyribonucleotides. However they are preferably oligonucleotides which are chemically modified on the level of the sugar radicals, the nucleobases, the phosphate groups and/or the backbone located in between, in order to increase for example the stability of the oligonucleotides in the cosmetic or dermatological preparation and/or in the skin, e.g. vis-à-vis a nucleolytic decomposition, in order to improve the penetration of the antisense oligonucleotides into the skin and the cell, in order to favourably influence the effectiveness of the antisense oligonucleotides and/or to improve the affinity to the sequence sections to be hybridized.

Oligonucleotides are preferred in which one or more phosphate groups are replaced by phosphothioate, methylphosphonate and/or phosphoramidate groups, such as e.g. N3′->P5′-phosphoramidate groups. Oligonucleotides in which phosphate groups are replaced by phosphothioate groups are particularly preferred. One or more of the phosphate groups of the oligonucleotide can be modified. In the case of a partial modification, terminal groups are preferably modified, but oligonucleotides in which all phosphate groups are modified are particularly preferred. This applies by analogy also to the modifications described below.

Preferred sugar modifications include the replacement of one or more ribose radicals of the oligonucleotide by morpholine rings (morpholine oligonucleotides) or with amino acids (peptide oligonucleotides). All ribose radicals of the oligonucleotide are preferably replaced by amino acid radicals and in particular morpholine radicals.

Morpholine oligonucleotides are particularly preferred in which the morpholine radicals are connected to one another via sulfonyl or preferably phosphoryl groups, as can be seen in Formula 1 or 2: 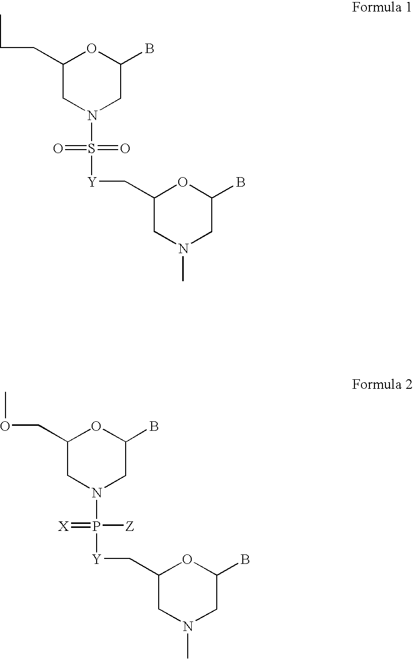
B stands for a modified or non-modified purine or pyrimidine base, preferably for adenine, cytosine, guanine or uracil,
X stands for O or S, preferably O,
Y stands for O or N—CH₃, preferably O,
Z stands for alkyl, O-alkyl, S-alkyl, NH₂, NH(alkyl), NH(O-alkyl), N(alkyl)₂, N(alkyl) (O-alkyl), preferably N(alkyl)₂, alkyl standing for linear or branched alkyl groups with 1 to 6, preferably 1 to 3, and particularly preferably 1 or 2 carbon atoms.

Formulae 1 and 2 each represent only a section of an oligonucleotide chain.

Morpholine oligonucleotides are quite particularly preferred in which the morpholine radicals are connected to one another via phosphoryl groups, as shown in Formula 2 in which X stands for O, Y for O and Z for N(CH₃)₂

Furthermore, the ribose or deoxyribose radicals can be modified by fluorine, alkyl or O-alkyl radicals. Examples of modifications are 2′-fluoro, 2′-alkyl, 2′-O-alkyl, 2′-O-methoxyethyl modifications, 5′-palmitate derivatives and 2′-O-methylribonucleotides. Unless otherwise stated, alkyl preferably stands for linear, branched or cyclic alkyl groups with 1 to 30, preferably 1 to 20, particularly preferably 1 to 10 and quite particularly preferably 1 to 6 carbon atoms. Branched and cyclic radicals naturally have at least 3 carbons, cyclic radicals with at least 5 and in particular at least 6 carbon atoms being preferred. Oligonucleotides which contain a-nucleosides can equally be used. Suitable base modifications are described e.g. in U.S. Pat. No. 6,187,578 and WO 99/53101, which are incorporated herein by reference. The synthesis of modified and non-modified oligonucleotides as well as further suitable possible modifications are described in the literature. The production of modified and non-modified oligonucleotides is now also offered by numerous companies as a service, morpholine oligonucleotides e.g. from Gene Tools, One Summerton Way, Philomath, OR 97370, USA; phosphothioate oligonucleotides e.g. from Biomol GmbH, WaidmannstraBe 35, 22769 Hamburg.

To increase stability and/or penetration, the oligonucleotides can also be used in encapsulated form, for example encapsulated in liposomes. In addition, they can also be stabilized by the addition of cyclodextrins.

The compositions according to the invention preferably contain 0.00001 to 10 wt.-%, particularly preferably 0.0003 to 3 wt.-% and quite particularly preferably 0.01 to 1.0 of the oligonucleotide or oligonucleotides according to the invention, relative to the overall mass of the composition.

The oligonucleotides and compositions are suitable for the treatment and prophylaxis of aging- and environmentally-triggered degenerative and deficitary conditions of the skin and of skin adnexa, such as hair and glands, in particular the symptoms described above. They are suitable for the cosmetic and therapeutic treatment of degenerative skin conditions which are caused by endogenous and exogenous factors, such as ozone and smoke and in particular UV radiation. The compositions according to the invention can prevent skin damage and can repair existing damage permanently and without the risk of side-effects.

The oligonucleotides according to the invention are particularly suitable for the prevention and treatment of skin changes due to age and skin changes which are caused by UV radiation in the connective tissue, such as e.g. skin changes which accompany biochemical, quantitative or qualitative changes in different dermal, extracellular proteins, in particular elastin, interstitial collagen and glycosaminoglycans. Wrinkling, slackness of the skin, loss of elasticity and mispigmentations (e.g. age marks) may primarily be named here.

The oligonucleotides and compositions are suitable for the prophylaxis and treatment of dryness, roughness of the skin, the formation of dry lines, reduced rehydration by sebaceous glands and an increased susceptibility to mechanical stress (tendency to crack), for the treatment of photodermatoses, the symptoms of senile xerosis, photoaging and other degenerative conditions which are associated with a decomposition of the connective tissue (collagen and elastin fibres and also glucosaminoglycans/hyaluronane) of the skin. “Photoaging” denotes the wrinkling, dryness and decreasing elasticity of the skin brought about by light and in particular UV radiation.

Due to their prophylactic action, the oligonucleotides and compositions are also outstandingly suitable for care of the skin.

The compositions according to the invention are also suitable for the treatment of skin damage caused by UV rays, e.g. the ultraviolet portion of solar radiation. UVB rays (290 to 320 nm) cause for example erythemas, sunburn or even burns of greater or lesser severity. UVA rays (320 to 400 nm) can cause irritations in light-sensitive skin and result in damage to the elastic and collagen fibres of the connective tissue, which causes the skin to age prematurely. In addition they are the cause of numerous phototoxic and photoallergic reactions. The oligonucleotides according to the invention are also suitable for the treatment of e.g. structural damage caused by UV rays and dysfunctions in the epidermis and dermis of the skin, such as for example visible vascular dilatations, such as telangiectasis and cuperosis, slackness of the skin and increase in wrinkles, local hyper-, hypo- and mispigmentations, such as e.g. age marks, and increased susceptibility to mechanical stress, e.g. tendency of the skin to crack.

Further fields of application of the compositions according to the invention are the treatment and prevention of age- and/or UV-induced collagen degeneration and also the decomposition of elastin and glycosaminoglycans; of degenerative skin conditions such as loss of elasticity and also atrophy of the epidermal and dermal cell layers, of constituents of the connective tissue, of rete pegs and capillary vessels) and/or the skin adnexa; of environmentally-triggered negative changes in the skin and the skin adnexa, e.g. caused by ultraviolet radiation, smoke, smog, reactive oxygen species, free radicals and similar; of deficitary, sensitive or hypoactive skin conditions or deficitary, sensitive or hypoactive skin adnexa conditions; the reduction in skin thickness; of skin slackness and/or skin tiredness; of changes in the transepidermal water loss and normal moisture content of the skin; of a change in the energy metabolism of healthy skin; of deviations from the normal cell-cell communication in the skin which can manifest themselves e.g. in wrinkling; of changes in the normal fibroblast and keratinocyte proliferation; of changes in the normal fibroblast and keratinocyte differentiation; of polymorphic actinodermatosis, vitiligo; of wound healing disorders; disturbances to the normal collagen, hyaluronic acid, elastin and glycosaminoglycan homeostasis; of increased activation of proteolytic enzymes in the skin, such as e.g. metalloproteinases.

According to the invention, compositions for topical applications are preferred. The compositions can be in all galenic forms which are usually used for a topical application, e.g. as solution, cream, ointment, lotion, shampoo, i.e. of the water-in-oil (W/O) emulsion type or of the oil-in-water (O/W) type, multiple emulsion, for example of the water-in-oil-in-water (W/o/W) type, or oil-in-water-in-oil (O/W/o) type, hydrodispersion or lipodispersion, Pickering emulsion, gel, stick or aerosol.

The cosmetic or medical treatment of the named indications is carried out as a rule by single or repeated application of the compositions according to the invention to the skin, preferably to the affected parts of the skin.

The compositions according to the invention are suitable in particular for cosmetic and therapeutic, i.e. in particular dermatological, application.

By cosmetic care of the skin is meant primarily that the natural function of the skin as a barrier against environmental influences (e.g. dirt, chemicals, microorganisms) and against the loss of the body's own substances (e.g. water, natural fats, electrolytes) is reinforced or restored. If this function is disrupted, increased resorption of toxic or allergenic substances or attack by microorganisms and consequently toxic or allergic skin reactions may result. The aim of skin care is further to compensate for the fat and water lost by the skin due to daily washing. This is particularly important when the natural regeneration capacity is insufficient. In addition, skin care products are to protect against environmental influences, in particular against sun and wind.

For cosmetic application, the compositions according to the invention therefore preferably contain components which are suitable for the named purposes. Such substances are known per se to a person skilled in the art. For example, one or more antisense oligonucleotides can be incorporated into customary cosmetic and dermatological preparations, which can be present in various forms.

According to a particularly preferred embodiment, the compositions according to the invention for cosmetic application are present as emulsion, e.g. in the form of a cream, a lotion, a cosmetic milk. These contain, in addition to the named oligonucleotides, further components such as e.g. fats, oils, waxes and/or other fatty substances, plus water and one or more emulsifiers such as are usually used for such a formulation type.

As a rule, emulsions contain a lipid or oil phase, an aqueous phase and preferably also one or more emulsifiers. Compositions are particularly preferred which also contain one or more hydrocolloids.

The compositions according to the invention preferably contain 0.001 to 35 wt.-%, particularly preferably 2 to 15 wt.-% emulsifier, 0.001 to 45 wt.-%, particularly preferably 10 to 25 wt.-% lipid and 10 to 95 wt.-%, particularly preferably 60 to 90 wt.-% water.

The lipid phase of the cosmetic or dermatological emulsions according to the invention can advantageously be chosen from the following substance group: (1) mineral oils, mineral waxes; (2) oils such as triglycerides of capric or caprylic acid, also natural oils such e.g. castor oil; (3) fats, waxes and other natural and synthetic fatty substances, preferably esters of fatty acids with alcohols of low C number, e.g. with isopropanol, propylene glycol or glycerol, or esters of fatty alcohols with alkanoic acids of low C number or with fatty acids; (4) alkyl benzoates; (5) silicone oils such as dimethylpolysiloxanes, diethylpolysiloxanes, diphenylpolysiloxanes and also mixed forms thereof.

Unless otherwise stated, by low C number is meant here preferably 1 to 5, particularly preferably 1 to 3 and quite particularly preferably 3 carbon atoms.

The oil phase of the emulsions of the present invention is advantageously chosen from the group of esters from saturated and/or unsaturated, branched or unbranched alkane carboxylic acids of a chain length of 3 to 30 C atoms and saturated and/or unsaturated, branched and/or unbranched alcohols of a chain length of 3 to 30 C atoms, from the group of esters from aromatic carboxylic acids and saturated and/or unsaturated, branched and/or unbranched alcohols of a chain length of 3 to 30 C atoms. Such ester oils can advantageously be chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and also synthetic, semi-synthetic and natural mixtures of such esters, e.g. jojoba oil.

Furthermore the oil phase can advantageously be chosen from the group of branched and unbranched hydrocarbons and waxes, silicone oils, dialkyl ethers, the group of saturated or unsaturated, branched or unbranched alcohols, and also the fatty acid triglycerides, namely the triglycerol esters of saturated and/or unsaturated, branched and/or unbranched alkane carboxylic acids of a chain length of 8 to 24, in particular 12-18 C atoms. The fatty acid triglycerides can for example advantageously be chosen from the group of synthetic, semi-synthetic and natural oils, e.g. olive oil, sunflower oil, soya oil, peanut oil, rape-seed oil, almond oil, palm oil, coconut oil, palm-kernel oil and more of this kind.

Any desired mixtures of such oil and wax components can also advantageously be used within the meaning of the present invention. It may also be advantageous where appropriate to use waxes, for example cetyl palmitate, as sole lipid component of the oil phase.

The oil phase is advantageously chosen from the group 2-ethylhexyl isostearate, octyl dodecanol, isotridecyl isononanoate, isoeicosane, 2-ethylhexyl cocoate, C₁₂₋₁₅ alkylbenzoate, caprylic-capric acid triglyceride, dicaprylyl ether.

Mixtures of C₁₂₋₁₅ alkylbenzoate and 2-ethylhexyl isostearate, mixtures of C₁₂₋₁₅ alkylbenzoate and isotridecyl isononanoate and also mixtures of C₁₂₋₁₅ alkylbenzoate, 2-ethylhexyl isostearate and isotridecyl isononanoate are particularly advantageous.

Of the hydrocarbons, paraffin oil, squalane and squalene are advantageously to be used within the meaning of the present invention.

The oil phase can advantageously also contain cyclic or linear silicone oils or consist entirely of such oils, it being preferred however to use an additional content of other oil phase components in addition to the silicone oil or silicone oils. Such silicones or silicone oils can be present as monomers which are characterized as a rule by structural elements, as follows:

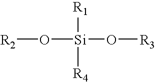

Linear silicones with several siloxyl units which can advantageously be used according to the invention are in general characterized by structural elements as follows: 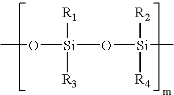
the silicon atoms being able to be substituted by the same or different alkyl radicals and/or aryl radicals which are represented here in generalized form by the radicals R₁-R₄ (in other words the number of different radicals is not necessarily restricted to 4). m can assume values of 2 to 200,000. Here, aryl preferably stands for phenyl, unless otherwise stated.

Cyclic silicones to be used advantageously according to the invention are generally characterized by structural elements, as follows: 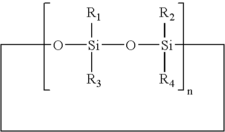
the silicon atoms being able to be substituted by the same or different alkyl radicals and/or aryl radicals which are represented here in generalized form by the radicals R₁-R₄ (in other words the number of different radicals is not necessarily restricted to 4). n can assume values of 3/2 to 20. Fractional values of n take into account that odd numbers of siloxyl groups can be present in the cycle.

Cyclomethicon (e.g. decamethylcyclopentasiloxane) is used advantageously as silicone oil according to the invention. But other silicone oils can also be used advantageously within the meaning of the present invention, for example undecamethylcyclotrisiloxane, polydimethylsiloxane, poly(methylphenylsiloxane), cetyldimethicon, behenoxydimethicon.

Mixtures of cyclomethicon and isotridecyl isononanoate and also of cyclomethicon and 2-ethylhexyl isostearate are also advantageous.

However it is also advantageous to choose silicone oils of similar constitution as the above-named compounds, the organic side chains of which are derivatized, are for example polyethoxylated and/or polypropoxylated. These include for example polysiloxane-polyalkyl-polyether copolymers such as cetyl-dimethicon-copolyol, (cetyl-dimethicon-copolyol (and) polyglyceryl-4-isostearate (and) hexyl laurate).

Mixtures of cyclomethicon and isotridecyl isononanoate, of cyclomethicon and 2-ethylhexyl isostearate are also particularly advantageous.

The aqueous phase of the preparations according to the invention can advantageously contain alcohols, diols or polyols of low C number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monethyl ether and analogous products, also alcohols of low C number, e.g. ethanol, isopropanol, 1,2-propanediol, glycerol and also in particular one or more thickening agents which can advantageously be chosen from the group silicone dioxide, aluminium silicates.

Preparations according to the invention present as emulsions preferably contain one or more emulsifiers. These emulsifiers can advantageously be chosen from the group of the non-ionic, anionic, cationic or amphoteric emulsifiers.

Non-ionic emulsifiers include (1) partial fatty acid esters and fatty acid esters of polyhydric alcohols and their ethoxylated derivatives (e.g. glyceryl monostearates, sorbitan stearates, glyceryl stearyl citrates, sucrose stearates); (2) ethoxylated fatty alcohols and fatty acids; (3) ethoxylated fatty amines, fatty acid amides, fatty acid alkanol amides; (4) alkylphenol polyglycol ethers (e.g. Triton X).

Anionic emulsifiers include soaps (e.g. sodium stearate); fatty alcohol sulfates; mono-, di- and trialkyl phosphonic acid esters and their ethoxylates.

Cationic emulsifiers include quaternary ammonium compounds with a long-chained aliphatic radical, e.g. distearyl dimonium chloride.

Amphoteric emulsifiers include alkylaminoalkanecarboxylic acids, betaines, sulfobetaines, imidazoline derivatives.

There are also naturally occurring emulsifiers, which include beeswax, wool wax, lecithin and sterols.

O/W emulsifiers can advantageously be chosen for example from the group of polyethoxylated or polypropoxylated or polyethoxylated and polypropoxylated products, e.g. fatty alcohol ethoxylates, ethoxylated wool wax alcohols, polyethylene glycol ethers of general formula R—O—(—CH₂—CH₂—O—)ₙ—R′, fatty acid ethoxylates of the general formula R—COO—(—CH₂—CH₂—O—)ₙ—H, etherified fatty acid ethoxylates of general formula R—COO—(—CH₂—CH₂—O—)ₙ—R′, esterified fatty acid ethoxylates of general formula R—COO—(—CH₂—CH₂—O—)ₙ—C(O)—R′, polyethylene glycol glycerol fatty acid esters, ethoxylated sorbitan esters, cholesterol ethoxylates, ethoxylated triglycerides, alkyl ether carboxylic acids of general formula R—O—(—CH₂—CH₂—O—)ₙ—CH₂—COOH, polyoxyethylene sorbitol fatty acid esters, alkyl ether sulfates of general formula R—O—(—CH₂—CH₂—O—)ₙ—SO₃—H, fatty alcohol propoxylates of general formula R—O—(—CH₂—CH(CH₃)—O—)ₙ—H, polypropylene glycol ethers of general formula R—O—(—CH₂—CH(CH₃)—O—)ₙ—R′, propoxylated wool wax alcohols, etherified fatty acid propoxylates, R—COO—(—CH₂—CH(CH₃)—O—)ₙ—R′, esterified fatty acid propoxylates of general formula R—COO—(—CH₂—CH(CH₃)—O—)ₙ—C(O)—R′, fatty acid propoxylates of general formula R—COO— (—CH₂—CH(CH₃)—O—)ₙ—H, polypropylene glycol glycerol fatty acid esters, propoxylated sorbitan esters, cholesterol propoxylates, propoxylated triglycerides, alkyl ether carboxylic acids of general formula R—O—(—CH₂—CH(CH₃)O—)ₙ—CH₂—COOH, alkyl ether sulfates or acids of general formula R—O—(—CH₂—CH(CH₃)—O—)ₙ—SO₃—H on which these sulfates are based, fatty alcohol ethoxylates/propoxylates of general formula R—O—Xₙ—Yₘ—H, polypropylene glycol ethers of general formula R—O—Xₙ—Yₘ—R′, etherified fatty acid propoxylates of general formula R—COO—Xₙ—Yₘ—R′, fatty acid ethoxylates/propoxylates of general formula R—COO—Xₙ—Yₘ—H.

In all cases the variables n and m each stand, independently of each other, for an integer from 1 to 40, preferably 5 to 30.

Particularly advantageously according to the invention, the polyethoxylated or polypropoxylated or polyethoxylated and polypropoxylated O/W emulsifiers used are chosen from the group of substances with HLB values from 11-18, quite particularly advantageously with HLB values from 14.5-15.5, provided the O/W emulsifiers have saturated radicals R and R′.

If the O/W emulsifiers have unsaturated radicals R and/or R′, or if isoalkyl derivatives are present, the preferred HLB value of such emulsifiers can also be lower or higher.

It is advantageous to choose the fatty alcohol ethoxylates from the group of the ethoxylated stearyl alcohols, cetyl alcohols, cetyl stearyl alcohols (cetearyl alcohols). Particularly preferred are:
polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth 16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20),
polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20),
polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20),
polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20),
polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15),
polyethylene glycol (12) lauryl ether (laureth-12), polyethylene glycol (12) isolauryl ether (isolaureth-12),
polyethylene glycol (13) cetyl stearyl ether (ceteareth-13), polyethylene glycol (14) cetyl stearyl ether (ceteareth-14), polyethylene glycol (15) cetyl stearyl ether (ceteareth-15), polyethylene glycol (16) cetyl stearyl ether (ceteareth-16), polyethylene glycol (17) cetyl stearyl ether (ceteareth-17), polyethylene glycol (18) cetyl stearyl ether (ceteareth-18), polyethylene glycol (19) cetyl stearyl ether (ceteareth-19), polyethylene glycol (20) cetyl stearyl ether (ceteareth-20).

It is furthermore advantageous to choose the fatty acid ethoxylates from the following group:
polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate,
polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

Sodium laureth-11-carboxylate can advantageously be used as ethoxylated alkyl ether carboxylic acid or its salt.

Sodium laureth 1-4 sulfate can advantageously be used as alkyl ether sulfate.

Polyethylene glycol (30) cholesteryl ether can advantageously be used as ethoxylated cholesterol derivative. Polyethylene glycol (25) soya sterol has also proved successful.

The polyethylene glycol (60) evening primrose glycerides can advantageously be used as ethoxylated triglycerides.

It is also advantageous to choose the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to choose the sorbitan esters from the group polyethylene glycol (20) sorbitan monolaurate, polyethylene glycol (20) sorbitan monostearate, polyethylene glycol (20) sorbitan isostearate, polyethylene glycol (20) sorbitan monopalmitate, polyethylene glycol (20) sorbitan monooleate.

There can be used as advantageous W/O emulsifiers:

fatty alcohols with 8 to 30 carbon atoms, monoglycerol esters of saturated and/or unsaturated, branched and/or unbranched alkane carboxylic acids of a chain length of 8 to 24, in particular 12-18 C atoms, diglycerol esters of saturated and/or unsaturated, branched and/or unbranched alkane carboxylic acids of a chain length of 8 to 24, in particular 12-18 C atoms, monoglycerol ethers of saturated and/or unsaturated, branched and/or unbranched alcohols of a chain length of 8 to 24, in particular 12-18 C atoms, diglycerol ethers of saturated and/or unsaturated, branched and/or unbranched alcohols of a chain length of 8 to 24, in particular 12-18 C atoms, propylene glycol esters of saturated and/or unsaturated, branched and/or unbranched alkane carboxylic acids of a chain length of 8 to 24, in particular 12-18 C atoms and also sorbitan esters of saturated and/or unsaturated, branched and/or unbranched alkane carboxylic acids of a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, saccharose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Preparations according to the invention present as emulsions also preferably contain one or more hydrocolloids. These hydrocolloids can advantageously be chosen from the group of gums, polysaccharides, cellulose derivatives, layered silicates, polyacrylates and/or other polymers.

Preparations according to the invention present as hydrogels contain one or more hydrocolloids. These hydrocolloids can advantageously be chosen from the above-named group.

Gums include plant or tree saps which harden in air and form resins or extracts from water plants. There can advantageously be chosen from this group within the meaning of the present invention for example gum arabic, carob seed powder, tragacanth, karaya, guar gum, pectin, gellan gum, carrageenan, agar, algins, chondrus, xanthan gum.

Also advantageous is the use of derivatized gums such as e.g. hydroxypropyl guar (Jaguar® HP 8).

Polysaccharides and derivatives include e.g. hyaluronic acid, chitin and chitosan, chondroitin sulfates, starch and starch derivatives.

Cellulose derivatives include e.g. methylcellulose, carboxymethylcellulose, hydroxyethylcellulose, hydroxypropylmethyl cellulose.

Layered silicates include naturally occurring and synthetic aluminas such as e.g. montmorillonite, bentonite, hectorite, laponite, magnesium aluminium silicates such as Veegum®. They can be used as such or in modified form such as e.g. stearylalkonium hectorites.

Silicas can also advantageously be used.

Polyacrylates include e.g. carbopol types from Goodrich (Carbopol 980, 981, 1382, 5984, 2984, EDT 2001 or Pemulen TR2).

Polymers include e.g. polyacrylamides (Sepigel 305), polyvinyl alcohols, PVP, PVP/VA copolymers, polyglycols.

According to a further preferred embodiment the oligonucleotides used according to the invention are introduced into aqueous systems or surfactant preparations for cleaning skin and hair.

The cosmetic preparations according to the invention also preferably contain, in addition to the named components, excipients such as they are usually used in such preparations, e.g. preservatives, bactericides, deodorants, antiperspirants, insect repellents, vitamins, anti-foaming agents, dyes, pigments with colouring action, thickening agents, plasticizers, moisturizing and/or moistening substances (moisturizers), or other customary constituents of a cosmetic formulation such as polyols, polymers, foam stabilizers, electrolytes, organic solvents or silicone derivatives, antioxidants and in particular UV absorbers.

Moisturizers are substances or substance mixtures which give cosmetic or dermatological preparations the property, after application or spreading on the surface of the skin, of reducing the moisture loss from the keratin layer (also called transepidermal water loss (TEWL)) and/or positively influencing the hydration of the keratin layer. Advantageous moisturizers within the meaning of the present invention are for example glycerol, lactic acid, pyrrolidone carboxylic acid and urea. It is furthermore particularly advantageous to use polymeric moisturizers from the group of polysaccharides which are soluble in water and/or swellable in water and/or gellable with the help of water. Particularly advantageous are for example hyaluronic acid and/or a fucose-rich polysaccharide which is filed in the Chemical Abstracts under the registration number 178463-23-5 and can be obtained e.g. under the name Fucogel 1000 from SOLABIA S.A.

When used as a moisturizer, glycerol is preferably used in a quantity of 0.05-30 wt.-%, particularly preferably 1-10%.

The cosmetic compositions can also advantageously contain one or more of the following natural active ingredients or a derivative thereof: alpha-liponic acid, phytoene, D-biotin, coenzyme Q10, alpha glycosyl rutin, carnitine, carnosine, natural and/or synthetic isoflavonoids, creatine, hop or hop-malt extract, taurine. Thus it transpired that active ingredients for positively influencing aging skin, which reduce the formation of wrinkles or else reduce existing wrinkles, such as bioquinones and in particular ubiquinone Q10, soya, creatinine, creatine, liponamide, or promote the restructuring of the connective tissue, such as isoflavone, can be very well used in the formulations according to the invention. It also transpired that the formulations are particularly suitable for combination with active ingredients to support skin functions in the case of dry skin, in particular age-dried skin, such as serinol and osmolytes, e.g. taurine. The incorporation of pigmentation modulators also proved advantageous. Here active ingredients are to be named which reduce the pigmentation of the skin and thus lead to a cosmetically desired brightening of the skin and/or reduce the occurrence of age marks and/or brighten existing age marks (tyrosine sulfate, dioic acid (8-hexadecene-1,16-dicarboxylic acid), liponic acid and liponamide, various liquorice extracts, kojic acid, hydroquinone, arbutin, fruit acids, in particular alpha-hydroxy acids (AHAs), bearberry (Uvae ursi), ursolic acid, ascorbic acid, green tea extract).

According to a particularly preferred version, the compositions according to the invention contain one or more UV absorbers. Preferred UV absorbers are those which absorb in the region of the UVB and/or UVA rays.

Numerous compounds for protection against UVB radiation are known which are derivatives of 3-benzylidene camphor, 4-aminobenzoic acid, cinnamic acid, salicylic acid, benzophenone and also 2-phenylbenzimidazole. Filters with an absorption maximum in the region of 308 nm are preferred, as the maximum erythemic effectiveness of sunlight lies here.

Advantageous UV-A filter substances within the meaning of the present invention are dibenzoylmethane derivatives, in particular 4-(tert.-butyl)-4′-methoxydibenzoylmethane (CAS no. 70356-09-1) which is sold by Givaudan under the trademark Parsol 1789® and by Merck under the trade name Eusolex® 9020.

The preparations according to the invention advantageously contain substances which absorb UV radiation in the UV-A and/or UV-B region, the overall quantity of filter substances being e.g. 0.1 wt.-% to 30 wt.-%, preferably 0.5 to 20 wt.-%, in particular 1.0 to 15.0 wt.-%, relative to the overall mass of the preparations, in order to provide cosmetic preparations which protect hair or skin against the whole range of ultraviolet radiation. They can also serve as sunscreens for hair or skin.

Further advantageous UV-A filter substances are phenylene-1,4-bis-(2-benzimidazyl)-3,3′-5,5′-tetrasulfonic acid 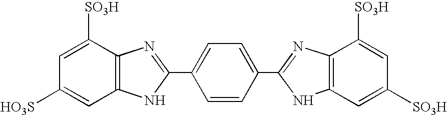
and its salts, in particular the corresponding sodium, potassium or triethanol ammonium salts, in particular phenylene-1,4-bis-(2-benzimidazyl)-3,3′-5,5′-tetrasulfonic acid-bis-sodium salt 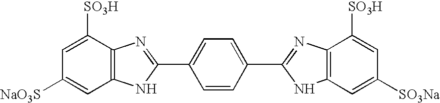
with the INCI name bisimidazylate which can be obtained for example under the trade name Neo Heliopan AP from Haarmann & Reimer.

Also advantageous are 1,4-di(2-oxo-10-sulfo-3-bornylidenemethyl)-benzene and its salts (in particular the corresponding 10-sulfato compounds, in particular the corresponding sodium, potassium or triethanol ammonium salt), which is also called benzene-1,4-di(2-oxo-3-bornylidenemethyl-10-sulfonic acid) and is characterized by the following structure:

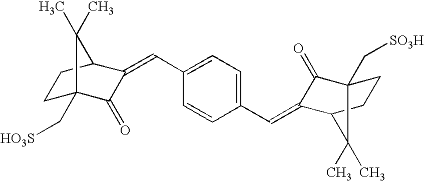

Advantageous UV filter substances within the meaning of the present invention are also so-called broadband filters, i.e. filter substances which absorb both UV-A and UV-B radiation.

Advantageous broadband filters or UV-B filter substances are for example bis-resorcinyl triazine derivatives with the following structure: 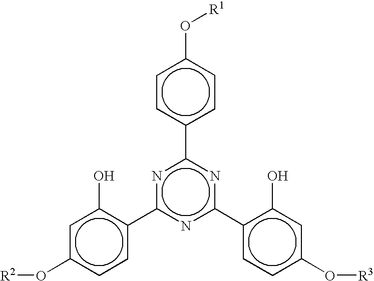
R¹, R₂ and R³ being chosen independently from one another from the group of branched and unbranched alkyl groups with 1 to 10 carbon atoms or representing a single hydrogen atom. Particularly preferred are 2,4-bis-{[4-(2-ethyl-hexyloxy)-2-hydroxy]-phenyl}-6-(4-methoxyphenyl)-1,3,5-triazine (INCI: Aniso Triazine), which can be obtained under the trade name Tinosorb® S from CIBA-Chemikalien GmbH, and 4,4′-4″-(1,3,5-triazine-2,4,6-triyltriimino)-tris-benzoic acid-tris (2-ethylhexylester), synonym: 2,4,6-tris-[anilino-(p-carbo-2′-ethyl-1′-hexyloxy)]-1,3,5-triazine (INCI: Octyl Triazone), which is sold by BASF Aktiengesellschaft under the trade name UVINUL® T 150.

Other UV filter substances which have the structural unit 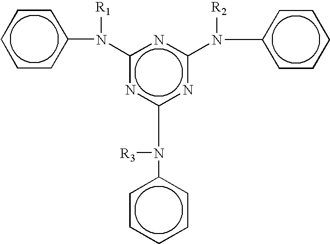
are also advantageous UV filter substances within the meaning of the present invention, for example the s-triazine derivatives described in the European unexamined patent application EP 570 838 A1, the chemical structure of which is reproduced by the generic formula 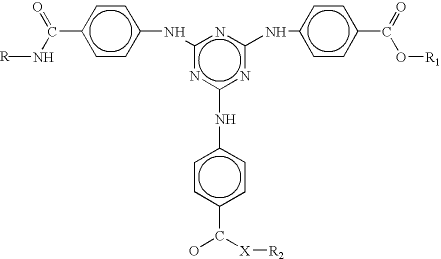
R representing a branched or unbranched C₁-C₁₈ alkyl radical, a C₅-C₁₂ cycloalkyl radical, optionally substituted by one or more C₁-C₄ alkyl groups,
X representing an oxygen atom or an NH group,
R₁ standing for a branched or unbranched C₁-C₁₈ alkyl radical, a C₅-C₁₂ cycloalkyl radical, optionally substituted by one or more C₁-C₄ alkyl groups, or a hydrogen atom, an alkali metal atom, an ammonium group or a group of the formula 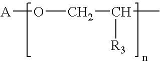
   in which
   A represents a branched or unbranched C₁-C₁₈ alkyl radical, a C₅-C₁₂ cycloalkyl or aryl radical, optionally substituted by one or more C₁-C₄ alkyl groups,
   R₃ represents a hydrogen atom or a methyl group,
   n represents a number from 1 to 10,
R₂ standing for a branched or unbranched C₁-C₁₈ alkyl radical, a C₅-C₁₂ cycloalkyl radical, optionally substituted by one or more C₁-C₄ alkyl groups if X represents the NH group, and
   a branched or unbranched C₁-C₁₈ alkyl radical, a C₅-C₁₂ cycloalkyl radical, optionally substituted by one or more C₁-C₄ alkyl groups, or a hydrogen atom, an alkali metal atom, an ammonium group or a group of the formula 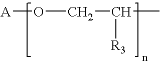
   in which
   A represents a branched or unbranched C₁-C₁₈ alkyl radical, a C₅-C₁₂ cycloalkyl or aryl radical, optionally substituted by one or more C₁-C₄ alkyl groups,
   R₃ represents a hydrogen atom or a methyl group,
   n represents a number from 1 to 10,
   if X represents an oxygen atom.

A particularly advantageous UV filter substance within the meaning of the present invention is also an unsymmetrically substituted s-triazine the chemical structure of which is represented by the formula 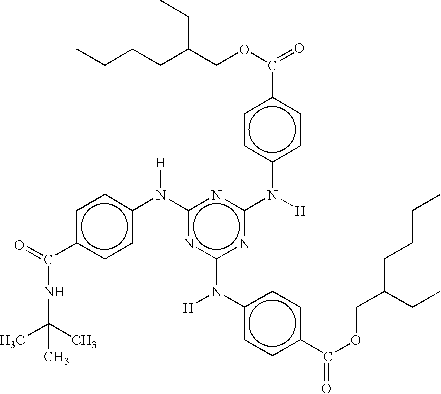
which is also called dioctylbutylamidotriazone (INCI: dioctylbutamido-triazone) and is available under the trade name UVASORB HEB from Sigma 3V.

Bis-resorcinyl triazine derivatives which can be used advantageously the chemical structure of which is reproduced by the generic formula 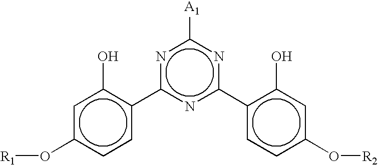
are also described in the European unexamined patent application EP 775 698, R₁, R₂ and A₁ representing very different organic radicals.

Also advantageous within the meaning of the present invention are 2,4-bis-{[4-(3-sulfonato)-2-hydroxypropyloxy)-2-hydroxy]-phenyl}-6-(4-methoxyphenyl)-1,3,5-triazine sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethylhexyloxy)-2-hydroxy]-phenyl}-6-[4-(2-methoxyethyl-carboxyl)-phenylamino]-1,3,5-triazine, 2,4-bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyloxy)-2-hydroxy]-phenyl}-6-(4-methoxyphenyl)-1,3,5-triazine and 2,4-bis-{[4-(1′,1′,1′,3′,5′,5′,5′-heptamethylsiloxy-2″-methyl-propyloxy)-2-hydroxy]-phenyl-6-(4-methoxyphenyl)-1,3,5-triazine.

An advantageous broadband filter within the meaning of the present invention is 2,2′-methylene-bis-(6-(2H-benzotriazol-2-yl)-4-(1,1,3,3-tetramethylbutyl)-phenol) [INCI: bisoctyltriazole] which is characterized by the chemical structural formula 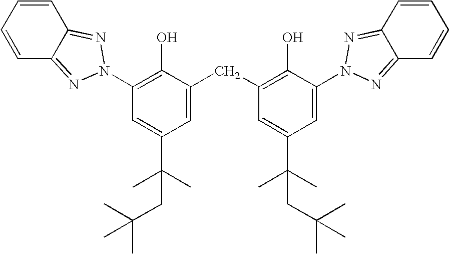
and can be obtained under the trade name Tinosorb® M from CIBA-Chemikalien GmbH.

An advantageous broadband filter within the meaning of the present invention is also 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS no.: 155633-54-8) with the INCI name drometrizole trisiloxane, which is characterized by the chemical structural formula

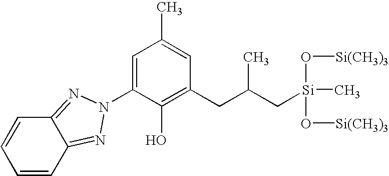

The UVB filters can be oil-soluble or water-soluble. Advantageous oil-soluble UVB filter substances are e.g.: 3-benzylidene camphor derivatives, preferably 3-(4-methylbenzylidene) camphor, 3-benzylidene camphor; 4-aminobenzoic acid derivatives, preferably 4-(dimethylamino)benzoic acid (2-ethylhexyl) ester, 4-(dimethylamino) benzoic acid amyl ester; 2,4,6-trianilino-(p-carbo-2′-ethyl-1′-hexyloxy)-1,3,5-triazine; esters of benzalmalonic acid, preferably 4-methoxybenzalmalonic acid di(2-ethylhexyl) ester; esters of cinnamic acid, preferably 4-methoxycinnamic acid (2-ethylhexyl) ester, 4-methoxycinnamic acid isopentyl ester; derivatives of benzophenone, preferably 2-hydroxy-4-methoxybenzophenone, 2-hydroxy-4-methoxy-4′-methylbenzophenone, 2,2′-dihydroxy-4-methoxybenzophenone and also UV filters bound to polymers.

Advantageous water-soluble UVB filter substances are e.g. salts of 2-phenylbenzimidazole-5-sulfonic acid, such as its sodium, potassium or its triethanol ammonium salt, and also sulfonic acid itself; sulfonic acid derivatives of 3-benzylidene camphor such as e.g. 4-(2-oxo-3-bornylidenemethyl)-benzenesulfonic acid, 2-methyl-5-(2-oxo-3-bornylidenemethyl) sulfonic acid and its salts.

A further light-protection filter substance which can be used advantageously according to the invention is ethylhexyl-2-cyano-3,3-diphenylacrylate (Octocrylene) which can be obtained from BASF under the name Uvinul® N539 and is characterized by the following structure:

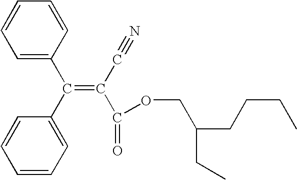

It can also be of considerable advantage to use polymer-bound or polymeric UV filter substances in preparations according to the present invention, in particular those such as are described in WO-A-92/20690.

It can also be advantageous where appropriate according to the invention to incorporate further UV-A and/or UV-B filters into cosmetic or dermatological preparations, for example certain salicylic acid derivatives such as 4-isopropylbenzyl salicylate, 2-ethylhexyl salicylate (=octyl salicylate), homomenthyl salicylate.

The list of the named UV filters which can be used within the meaning of the present invention is not of course intended to be limitative.

The compositions according to the invention can also be antioxidants to protect the cosmetic preparation itself or to protect the constituents of the cosmetic preparations against harmful oxidation processes.

The antioxidants are advantageously chosen from the group consisting of amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotinoids, carotenes (e.g. α-carotene, β-carotene, lycopine) and their derivatives, aurothioglucose, propylthiouracil and other thiols (e.g. thioredoxin, glutathion, cysteine, cystine, cystamine and its glycosyl, N-acetyl, methyl, ethyl, propyl, amyl, butyl and lauryl, palmitoyl, oleyl, γ-linoleyl, cholesteryl and glyceryl esters) and also their salts, dilauryl thiodipropionate, distearyl thiodipropionate, thiodipropionic acid and its derivatives (esters, ethers, peptides, lipids, nucleotides, nucleosides and salts) and also sulfoximine compounds (e.g. buthionine sulfoximines, homocysteine sulfoximine, buthionine sulfones, penta-, hexa-, heptathionine sulfoximine) in very low compatible dosages (e.g. pmol to μmol/kg), furthermore (metal) chelators (e.g. α-hydroxy fatty acids, palmitic acid, phytinic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linoleic acid, linolic acid, oleic acid), folic acid and its derivatives, alanine diacetic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and also coniferyl benzoate of gum benzoin, rutinic acid and its derivatives, ferulic acid and its derivatives, butylhydroxytoluene, butylhydroxyanisole, nordihydroguaiac resin acid, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenium methionine), stilbenes and their derivatives (e.g. stilbene oxide, transstilbene oxide) and the derivatives suitable according to the invention (salts, esters, ethers, sugars, nucleotides, nucleosides, peptides and lipids) of these named active ingredients.

The quantity of antioxidants (one or more compounds) in the preparations is preferably 0.001 to 30 wt.-%, particularly preferably 0.05-20 wt.-%, in particular 1-10 wt.-% relative to the overall mass of the preparation.

Cosmetic and therapeutic preparations according to the invention also advantageously contain inorganic pigments based on metal oxides and/or other metal compounds poorly soluble or insoluble in water, in particular the oxides of titanium (TiO₂), zinc, (ZnO), iron (e.g. Fe₂O₃), zirconium (ZrO₂), silicone (SiO₂), manganese (e.g. MnO), aluminium (Al₂O₃), cerium (e.g. Ce₂O₃), mixed oxides of the corresponding metals and also mixtures of such oxides. TiO₂-based pigments are particularly preferred.

It is particularly advantageous within the meaning of the present invention, if not essential, for the inorganic pigments to be present in hydrophobic form, i.e., for their surface to have been hydrophobically treated. This surface treatment can consist of the pigments being provided with a thin hydrophobic layer using processes known per se.

Such a process consists for example of producing the hydrophobic surface layer according to a reaction as per
n TiO₂+m (RO)₃ Si—R′->n TiO₂ (surf.)
   n and m are stoichiometric parameters to be applied as wished, R and R′ the desired organic radicals. Hydrophobized pigments prepared for example by analogy to DE-OS 33 14 742 are advantageous.

Advantageous TiO₂ pigments can be obtained for example under the trade names MT 100 T from TAYCA, also M 160 from Kemira and also T 805 from Degussa.

Preparations according to the invention can also, particularly if crystalline or microcrystalline solids, for example inorganic micropigments, are to be incorporated into the preparations according to the invention, contain anionic, non-ionic and/or amphoteric surfactants.

Surfactants are amphiphilic substances which can dissolve organic, non-polar substances in water.

The hydrophilic portions of a surfactant molecule are mostly polar functional groups, for example —COO⁻, —OSO₃²⁻, —SO₃⁻, whereas the hydrophobic parts are as a rule non-polar hydrocarbon radicals. Surfactants are generally classified according to the type and charge of the hydrophilic molecule part. A distinction can be made between four groups, namely anionic surfactants, cationic surfactants, amphoteric surfactants and non-ionic surfactants.

As functional groups, anionic surfactants usually have carboxylate, sulfate or sulfonate groups. In aqueous solution, they form negatively charged organic ions in an acid or neutral environment. Cationic surfactants are almost exclusively characterized by the presence of a quaternary ammonium group. In aqueous solution, they form positively charged organic ions in an acid or neutral environment. Amphoteric surfactants contain both anionic and cationic groups and accordingly behave as anionic or cationic surfactants in aqueous solution, depending on the pH value. They have a positive charge in a strongly acid environment and a negative charge in an alkaline environment. In the neutral pH range, on the other hand, they are zwitterionic, as the following example is intended to illustrate:
pH=2 RNH₂⁺CH₂CH₂COOH X⁻
   (X−=any anion, e.g. Cl⁻)
pH=7 RNH₂⁺CH₂CH₂COO⁻
pH=12 RNHCH₂CH₂COO⁻ B⁺
   (B⁺=any cation, e.g. Na⁺)

Polyether chains are typical of non-ionic surfactants. Non-ionic surfactants do not form ions in an aqueous medium.

Anionic surfactants which can be used advantageously are:
acylamino acids (and their salts), such as (1) acyl glutamates, for example sodium acyl glutamate, Di-TEA-palmitoyl aspartate and sodium caprylic/capric glutamate; (2) acyl peptides, for example palmitoyl-hydrolyzed lactoprotein, sodium cocoyl-hydrolyzed soya protein and sodium/potassium cocoyl-hydrolyzed collagen; (3) sarcosinates, for example myristoyl sarcosine, TEA-lauroyl sarcosinate, sodium lauroyl sarcosinate and sodium cocoyl sarcosinate; (4) taurates, for example sodium lauroyl taurate and sodium methyl cocoyl taurate; (5) acyl lactylates, such as lauroyl lactylate and caproyl lactylate; (6) alaninates;
carboxylic acids and derivatives, such as for example lauric acid, aluminium stearate, magnesium alkanolate and zinc undecylenate; ester carboxylic acids, for example calcium stearoyl lactylate; laureth-6 citrate and sodium PEG-4 lauramide carboxylate; ether carboxylic acids, for example sodium laureth-13 carboxylate and sodium PEG-6 cocamide carboxylate;
carboxylic acids, ester carboxylic acids and ether carboxylic acids preferably contain 1 to 50 and in particular 2 to 30 carbon atoms.

Phosphoric acids and salts, such as for example DEA-oleth-10-phosphate and dilaureth-4 phosphate;
sulfonic acids and salts, such as (1) acyl isethionates, e.g. sodium/ammonium cocoyl isethionate; (2) alkylaryl sulfonates; (3) alkyl sulfonates, for example sodium cocomonoglyceride sulfate, sodium C₁₂₋₁₄ olefin sulfonate, sodium lauryl sulfoacetate and magnesium PEG-3 cocamide sulfate; (4) sulfosuccinates, for example dioctyl sodium sulfosuccinate, disodium laureth sulfosuccinate, disodium lauryl sulfosuccinate and disodium undecyleneamido-MEA sulfosuccinate;
sulfuric acid esters, such as (1) alkyl ether sulfate, for example sodium, ammonium, magnesium, MIPA, TIPA laureth sulfate, sodium myreth sulfate and sodium C₁₂₋₁₃ pareth sulfate; (2) alkyl sulfates, for example sodium, ammonium and TEA lauryl sulfate.

Cationic surfactants which can be used advantageously are alkylamines, alkylimidazoles, ethoxylated amines and quaternary surfactants and also esterquats.

Quaternary surfactants contain at least one N atom which is covalently bound to 4 alkyl or aryl groups. This leads, irrespective of the pH value, to a positive charge. Alkylbetaine, alkylamidopropylbetaine and alkylamidopropylhydroxysulfaine are advantageous. The cationic surfactants used according to the invention can also preferably be chosen from the group of the quaternary ammonium compounds, in particular benzyltrialkyl ammonium chlorides or bromides, such as for example benzyldimethylstearyl ammonium chloride, also alkyltrialkyl ammonium salts, for example cetyltrimethyl ammonium chloride or bromide, alkyldimethylhydroxyethyl ammonium chlorides or bromides, dialkyldimethyl ammonium chlorides or bromides, alkylamide ethyltrimethyl ammonium ether sulfates, alkylpyridinium salts, for example lauryl or cetylpyrimidinium chloride, imidazoline derivatives and compounds with a cationic character such as amine oxides, for example alkyldimethylamine oxides or alkylaminoethyldimethylamine oxides. Cetyltrimethyl ammonium salts in particular are advantageously to be used.

Amphoteric surfactants which can be used advantageously are (1) acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate; (2) N-alkylamino acids, for example aminopropylalkyl glutamide, alkylamino propionic acid, sodium alkylimidodipropionate and lauroamphocarboxyglycinate.

Non-ionic surfactants which can be used advantageously are (1) alcohols; (2) alkanol amides, such as MEA/DEA/MIPA cocamides; (3) amine oxides, such as cocoamidopropylamine oxide; (4) esters which form through esterification of carboxylic acids with ethylene oxide, glycerol, sorbitan or other alcohols; (5) ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkylpolyglycosides such as lauryl glucoside, decyl glycoside and coco glycoside; (6) sucrose esters, ethers; (7) polyglycerol esters, diglycerol esters, monoglycerol esters; (8) methyl glucose esters, esters of hydroxy acids.

The use of a combination of anionic and/or amphoteric surfactants with one or more non-ionic surfactants is also advantageous.

The surfactant can be present in the preparations according to the invention in a concentration between 1 and 95 wt.-%, relative to the overall mass of the preparations.

Preparations for medical application are no different in their composition from the cosmetic products and can likewise contain the above named substances. They differ from the latter primarily in that they must undergo a special approval procedure.

The invention is explained in more detail below using embodiments. All the numerical values in the examples relate to wt.-% unless otherwise stated.

### EXAMPLES

### Example 1

### Preparation of PIT Emulsions

By mixing the components given in the table, phase inversion temperature emulsions (PIT emulsions) of the composition which is likewise given were prepared.

TABLE 1
PIT Emulsions

| | Emulsion No. | | | | |
|---|---|---|---|---|---|
| | 1 | 2 | 3 | 4 | 5 |
| Self-emulsifying | 0.50 | | 3.00 | 2.00 | 4.00 |
| glycerol monostearate | | | | | |
| Polyoxyethylene (12) | | 5.00 | | 1.00 | 1.50 |
| cetyl stearyl ether | | | | | |
| Polyoxyethylene (20) | | | | 2.00 | |
| cetyl stearyl ether | | | | | |
| Polyoxyethylene (30) | 5.00 | | 1.00 | | |
| cetyl stearyl ether | | | | | |
| Stearyl alcohol | | | 3.00 | | 0.50 |
| Cetyl alcohol | 2.50 | 1.00 | | 1.50 | |
| 2-ethylhexyl | | | | 5.00 | 8.00 |
| methoxycinnamate | | | | | |
| 2,4-bis-(4-(2-ethyl- | | 1.50 | | 2.00 | 2.50 |
| hexyloxy)2-hydroxyl)- | | | | | |
| phenyl)-6-(4- | | | | | |
| methoxyphenyl)-1,3,5)- | | | | | |
| triazine | | | | | |
| 1-(4-tert-butylphenyl)- | | | 2.00 | | |
| 3-(4-methoxyphenyl)- | | | | | |
| 1,3-propanedione | | | | | |
| Diethylhexylbutamido- | 1.00 | 2.00 | | 2.00 | |
| triazone | | | | | |
| Ethylhexyltriazone | 4.00 | | 3.00 | 4.00 | |
| 4-methylbenzylidene | | 4.00 | | | 2.00 |
| camphor | | | | | |
| Octocrylene | | 4.00 | | | 2.50 |
| Phenylene-1,4-bis- | | | 0.50 | | 1.50 |
| (monosodium, 2- | | | | | |
| benzimidazyl-5,7- | | | | | |
| disulfonic acid) | | | | | |
| Phenylbenzimidazole | 0.50 | | | 3.00 | |
| sulfonic acid | | | | | |
| C12-15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol- | 5.00 | | | 6.00 | |
| dicaprylate/-dicaprate | | | | | |
| Dicaprylyl carbonate | | | 6.00 | | 2.00 |
| Dimethicon | | 0.50 | 1.00 | | |
| polydimethylsiloxane | | | | | |
| Phenylmethyl- | 2.00 | | | 0.50 | 0.50 |
| polysiloxane | | | | | |
| Shea butter | | 2.00 | | | 0.50 |
| PVP hexadecene | 0.50 | | | 0.50 | 1.00 |
| copolymer | | | | | |
| Glycerol | 3.00 | 7.50 | 5.00 | 7.50 | 2.50 |
| Tocopherol acetate | 0.50 | | 0.25 | | 1.00 |
| MMP1 antisense | 0.10 | 0.10 | | 0.10 | 0.10 |
| oligonucleotide (SEQ ID | | | | | |
| NO 7) | | | | | |
| Preservative | q.s | q.s | q.s | q.s | q.s |
| Ethanol | 3.00 | 2.00 | 1.50 | | 1.00 |
| Perfume | q.s | q.s | q.s | q.s | q.s |
| Water | ad. | ad. | ad. | ad. | ad. |
| | 100 | 100 | 100 | 100 | 100 |

Analogous PIT formulations were obtained by using elastase (SEQ ID NO 19) or hyaluronidase (SEQ ID NO 31) antisense oligonucleotides or 0.1 wt.-% of a mixture of equal parts of anti-SEQ ID NO 7, anti-SEQ ID NO 19 and anti-SEQ ID NO 31.

The term MMP1 antisense oligonucleotide (SEQ ID NO 7) or anti-SEQ ID NO 7 denotes an anti-MMP1 oligonucleotide which hybridizes with the sequence SEQ ID NO 7. The other names used in this and in the other examples for the oligonucleotides used are to be understood analogously.

### Example 2

### Preparation of Creams Based on Oil-in-Water Emulsions

By mixing the components given in the table, creams the composition of which is likewise given were prepared.

TABLE 2
O/W Creams

| | Cream No | | | | |
|---|---|---|---|---|---|
| | 1 | 2 | 3 | 4 | 5 |
| Glyceryl stearate citrate | | | 2.00 | | 2.00 |
| Self-emulsifying glyceryl | 4.00 | 3.00 | | | |
| stearate | | | | | |
| PEG-40 stearate | 1.00 | | | | |
| Polyglyceryl-3- | | | | 3.00 | |
| methylglucose-distearate | | | | | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetyl stearyl alcohol | | | | | 2.00 |
| Caprylic/capric | 5.00 | 3.00 | 4.00 | 3.00 | 3.00 |
| triglyceride | | | | | |
| Octyl dodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-methylbenzylidene | | | 1.00 | | |
| camphor | | | | | |
| 1,4-tert-butylphenyl)-3- | | | 0.50 | | |
| (4-methoxyphenyl)-1,3- | | | | | |
| propanedione | | | | | |
| MMP1 antisense | 0.10 | 0.10 | 0.10 | 0.10 | 0.10 |
| oligonucleotides | | | | | |
| (SEQ ID NO 2) | | | | | |
| Tocopherol | 0.1 | | | | 0.20 |
| Biotin | | | 0.05 | | |
| Ethylenediamine- | 0.1 | | 0.10 | 0.1 | |
| tetraacetic | | | | | |
| acid trisodium | | | | | |
| Preservative | q.s | q.s | q.s | q.s | q.s |
| Polyacrylic acid | 3.00 | 0.1 | | 0.1 | 0.1 |
| Caustic soda 45% | q.s | q.s | q.s | q.s | q.s |
| Glycerol | 5.00 | 3.00 | 4.00 | 3.00 | 3.00 |
| Butylene glycol | | 3.00 | | | |
| Perfume | q.s | q.s | q.s | q.s | q.s |
| Water | ad. | ad. | ad. | ad. | ad. |
| | 100 | 100 | 100 | 100 | 100 |
| | Cream No. | | | | |
| | 6 | 7 | 8 | 9 | 10 |
| Glyceryl stearate citrate | | 2.00 | 2.00 | | |
| Self-emulsifying | 5.00 | | | | |
| glycerylsteareate | | | | | |
| Stearic acid | | | | 2.50 | 3.50 |
| Stearyl alcohol | 2.00 | | | | |
| Cetyl alcohol | | | | 3.00 | 4.50 |
| Cetyl stearyl alcohol | | 3.00 | 1.00 | | 0.50 |
| C12-15 alkyl benzoate | | 2.00 | 3.00 | | |
| Caprylic/capric | 2.00 | | | | |
| triglyceride | | | | | |
| Octyl dodecanol | 2.00 | 2.00 | | 4.00 | 6.00 |
| Dicaprylyl ether | | | | | |
| Liquid paraffin | | 4.00 | 2.00 | | |
| Cyclic | | | | 0.50 | 2.00 |
| dimethylpolysiloxane | | | | | |
| Dimethicon | 2.00 | | | | |
| polydimethylsiloxane | | | | | |
| Titanium dioxide | 2.00 | | | | |
| 4-methylbenzylidene | 1.00 | | | | 1.00 |
| camphor | | | | | |
| 1-(4-tert-butylphenyl)-3- | 0.50 | | | | 0.50 |
| (4-methoxyphenyl)-1,3- | | | | | |
| propanedione | | | | | |
| MMP1 antisense | 0.10 | 0.10 | 0.10 | 0.10 | 0.10 |
| oligonucleotide | | | | | |
| (SEQ ID NO 2) | | | | | |
| Tocopherol | | | | | 0.05 |
| Ethylenediamine- | | | 0.20 | | 0.20 |
| tetraacetic | | | | | |
| acid trisodium | | | | | |
| Preservative | q.s | q.s | q.s | q.s | q.s |
| Xanthan gum | | | 0.20 | | |
| Polyacrylic acid | 0.15 | 0.1 | | 0.05 | 0.05 |
| Caustic soda 45% | q.s | q.s | q.s | q.s | q.s |
| Glycerol | 3.00 | | 3.00 | 5.00 | 3.00 |
| Butylene glycol | | 3.00 | | | |
| Ethanol | | 3.00 | | 3.00 | |
| Perfume | q.s | q.s | q.s | q.s | q.s |
| Water | ad. | ad. | ad. | ad. | ad. |
| | 100 | 100 | 100 | 100 | 100 |

Analogous creams were obtained by using MMP1 antisense oligonucleotides (SEQ ID NO 5) or (SEQ ID NO 6) or 0.1 wt.-% of a mixture of equal parts of anti-SEQ ID NO 2, anti-SEQ ID NO 5 and anti-SEQ ID NO 6.

### Example 3

### Preparation of Water-In-Oil Emulsions

By mixing the components given in the table, water-in-oil emulsions, the composition of which is also given, were prepared.

TABLE 3
W/O Emulsions

| Emulsion No. | | | | | |
|---|---|---|---|---|---|
| Cetyldimethicon copolyol | | .50 | | 4.00 | |
| Polyglyceryl-2- | .00 | | | | 4.50 |
| dipolyhydroxystearate | | | | | |
| PEG-30- | | | 5.00 | | |
| dipolyhydroxystearate | | | | | |
| 2-ethylhexyl | | .00 | | 5.00 | 4.00 |
| methoxycinnamate | | | | | |
| 2,4-bis-(4-(2-ethyl- | .00 | .50 | | 2.00 | 2.50 |
| hexyloxy)-2-hydroxyl)- | | | | | |
| phenyl)-6-(4- | | | | | |
| methoxyphenyl)-(1,3,5)- | | | | | |
| triazine | | | | | |
| 1-(4-tert-butylphenyl)- | | | 2.00 | 1.00 | |
| 3-(4-methoxyphenyl)-1,3- | | | | | |
| propanedione | | | | | |
| Diethylhexylbutamido- | .00 | .00 | | | 3.00 |
| triazone | | | | | |
| Ethylhexyl triazone | | | 3.00 | 4.00 | |
| 4-methylbenzylidene | | .00 | | 4.00 | 2.00 |
| camphor | | | | | |
| Octocrylene | .00 | .50 | 4.00 | | 2.50 |
| Diethylhexylbutamido- | .00 | | | 2.00 | |
| triazone | | | | | |
| Phenylene-1-4-bis- | .00 | .00 | 0.50 | | |
| monosodium,2- | | | | | |
| benzimidazyl-5,7- | | | | | |
| disulfonic acid) | | | | | |
| Phenylbenzimazole | .50 | | | 3.00 | 2.00 |
| sulfonic acid | | | | | |
| Titanium dioxide | | .00 | 1.50 | | 3.00 |
| Zinc oxide | .00 | .00 | 2.00 | 0.50 | |
| Liquid paraffin | | | 10.0 | | 8.00 |
| C12-15 alkyl benzoate | | | | 9.00 | |
| Dicaprylyl ether | 0.00 | | | | 7.00 |
| Butylene-glycol- | | | 2.00 | 8.00 | 4.00 |
| dicaprylate/-dicaprate | | | | | |
| Dicaprylyl carbonate | .00 | | 6.00 | | |
| Dimethicon | | .00 | 1.00 | 5.00 | |
| polydimethylsiloxane | | | | | |
| Phenylmethylpolysiloxane | .00 | 5.00 | | | 2.00 |
| Shea butter | | | 3.00 | | |
| PVP hexadecene copolymer | .50 | | | 0.50 | 1.00 |
| Octoxyglycerol | | .30 | 1.00 | | 0.50 |
| Glycerol | .00 | .50 | | 7.50 | 2.50 |
| Glycine soya | | .00 | 1.50 | | |
| Magnesium sulfate | .00 | .50 | | 0.50 | |
| Magnesium chloride | | | 1.00 | | 0.70 |
| Tocopherol acetate | .50 | | 0.25 | | 1.00 |
| Elastase antisense | .10 | .10 | 0.10 | 0.10 | 0.10 |
| oligonucleotide (SEQ ID | | | | | |
| NO 15) | | | | | |
| Preservative | .s | .s | q.s | q.s | q.s |
| Ethanol | .00 | | 1.50 | | 1.00 |
| Perfume | .s | .s | q.s | q.s | q.s |
| Water | d. | d. | ad. | ad. | ad. |
| | 100 | 100 | 100 | 100 | 100 |

Emulsion No.

| | 6 | 7 |
|---|---|---|
| Polyglyceryl-2- | 4.00 | 5.00 |
| dipolyhydroxystearate | | |
| PEG-30- | | |
| dipolyhydroxystearate | | |
| Lanolin alcohol | 0.50 | 1.50 |
| Isohexadecane | 1.00 | 2.00 |
| Myristyl-myristate | 0.50 | 1.50 |
| Vaseline | 1.00 | 2.00 |
| 1-(4-tert-butylphenyl)- | 0.50 | 1.50 |
| 3-(4-methoxyphenyl)- | | |
| 1,3-propanedione | | |
| 4-methylbenzylidene | 1.00 | 3.00 |
| camphor | | |
| Butylene-glycol- | 4.00 | 5.00 |
| dicaprylate/-dicaprate | | |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Elastase antisense | 0.10 | 0.10 |
| oligonucleotide (SEQ ID | | |
| NO 15) | | |
| Trisodium EDTA | 0.20 | 0.20 |
| Preservative | q.s | q.s |
| Ethanol | | 3.00 |
| Perfume | q.s | q.s |
| Water | ad. 100 | ad. 100 |

Analogous emulsions were obtained by using elastase (SEQ ID No. 18) or hyaluronidase (SEQ ID NO 22) antisense oligonucleotides or 0.1 wt.-% of a mixture of equal parts of anti-SEQ ID NO 15, anti-SEQ ID NO 18 and anti-SEQ ID NO 22.

### Example 4

### Preparation of Hydrodispersions

By mixing the components given in the table, hydrodispersions, the composition of which is also given, were prepared.

TABLE 4
Hydrodispersions

| | Dispersion No. | | | | |
|---|---|---|---|---|---|
| | 1 | 2 | 3 | 4 | 5 |
| Polyoxyethylene (20) | 1.00 | | | 0.5 | |
| cetyl stearyl ether | | | | | |
| Cetyl alcohol | | | 1.00 | | |
| Sodium polyacrylate | | 0.20 | | 0.30 | |
| Acrylate/C10-30 alkyl- | 0.50 | | 0.40 | 0.10 | 0.10 |
| acrylate cross-polymer | | | | | |
| Xanthan gum | | 0.30 | 0.15 | | 0.50 |
| 2-ethylhexyl | | | | 5.00 | 8.00 |
| methoxycinnamate | | | | | |
| 2,4-bis-(4-(2-ethyl- | | 1.50 | | 2.00 | 2.50 |
| hexyloxy-)2-hydroxyl)- | | | | | |
| phenyl)-6-(4- | | | | | |
| methoxyphenyl)-(1,3,5)- | | | | | |
| triazine | | | | | |
| 1-(4-tert-butylphenyl)- | 1.00 | | 2.00 | | |
| 3-(4-methoxyphenyl)-1,3- | | | | | |
| propanedione | | | | | |
| Diethylhexylbutamido- | | 2.00 | | 2.00 | 1.00 |
| triazone | | | | | |
| Ethylhexyl triazone | 4.00 | | 3.00 | 4.00 | |
| 4-methylbenzylidene | 4.00 | 4.00 | | | 2.00 |
| camphor | | | | | |
| Octocrylene | | 4.00 | 4.00 | | 2.50 |
| Phenylene-1,4-bis- | 1.00 | | 0.50 | | 2.00 |
| (monosodium,2- | | | | | |
| benzimidazyl-5,7- | | | | | |
| disulfonic acid | | | | | |
| Phenylbenzimidazole | 0.50 | | | 3.00 | |
| sulfonic acid | | | | | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol- | 4.00 | | 2.00 | 6.00 | |
| dicaprylate/-dicaprate | | | | | |
| Dicaprylyl carbonate | | 2.00 | 6.00 | | |
| Dimethicon | | 0.50 | 1.00 | | |
| polydimethylsiloxane | | | | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Hyaluronidase antisense | 0.10 | 0.10 | 0.10 | 0.10 | 0.10 |
| oligonucleotide (SEQ ID | | | | | |
| NO 26) | | | | | |
| Preservative | q.s | q.s | q.s | q.s | q.s |
| Ethanol | 3.00 | 2.00 | 1.50 | | 1.00 |
| Perfume | q.s | q.s | q.s | q.s | q.s |
| Water | ad. | ad. | ad. | ad. | ad. |
| | 100 | 100 | 100 | 100 | 100 |

Analogous dispersions were obtained by using hyaluronidase antisense oligonucleotides (SEQ ID NO 32) or (SEQ ID NO 33) or 0.1 wt.-% of a mixture of equal parts of anti-SEQ ID NO 26, anti-SEQ ID NO 32 and anti-SEQ ID No. 33.

### Example 5

### Preparation of a Gel Cream

By mixing the components given in the table, a gel cream, the composition of which is also given, was prepared. The pH value of the gel cream was then set to 6.0.

TABLE 5
Gel cream

| | |
|---|---|
| Acrylate/C10-30 | 0.40 |
| alkylacrylate cross- | |
| polymer | |
| Polyacrylic acid | 0.20 |
| Xanthan gum | 0.10 |
| Cetearyl alcohol | 3.00 |
| C12-15 alkyl benzoate | 4.00 |
| Caprylic/Capric | 3.00 |
| triglyceride | |
| Cyclic | 5.00 |
| dimethylpolysiloxane | |
| MMP1 antisense | 0.10 |
| oligonucleotide (SEQ ID | |
| NO 13) | |
| Glycerol | 3.00 |
| Sodium hydroxide | q.s |
| Preservative | q.s |
| Perfume | q.s |
| Water | ad. 100.0 |
| pH value set to 6.0 | |

Analogous gel creams were obtained by using elastase (SEQ ID NO 16) or (SEQ ID NO 20) or hyaluronidase (SEQ ID NO 25) antisense oligonucleotides or 0.1 wt.-% of a mixture of equal parts of anti-SEQ ID NO 13, anti-SEQ ID NO 16, anti-SEQ ID NO 20 and anti-SEQ ID NO 25.

### Example 6

### Preparation of a Cream on the Basis of a Water-In-Oil Emulsion

By mixing the components given in the table, a cream, the composition of which is also given, was prepared on the basis of a water-in-oil dispersion.

TABLE 6
W/O-Cream

| | |
|---|---|
| Polyglyceryl-3- | 3.50 |
| diisostearate | |
| Glycerol | 3.00 |
| Polyglyceryl-2- | 3.50 |
| dipolyhydroxystearate | |
| MMP1 antisense | 0.10 |
| oligonucleotide (SEQ ID | |
| NO 10) | |
| Preservative | q.s |
| Perfume | q.s |
| Water | ad. 100.0 |
| Magnesium sulfate | 0.6 |
| Isopropyl stearate | 2.0 |
| Caprylyl ether | 8.0 |
| Cetearyl isononanoate | 6.0 |

Analogous creams were obtained by using MMP1 (SEQ ID NO 9) elastase (SEQ ID NO 17) or hyaluronidase (SEQ ID NO 30) antisense oligonucleotides or 0.1 wt.-% of a mixture of equal parts of these antisense oligonucleotides.

### Example 7

### Preparation of a Cream on the Basis of a Water-In-Oil-In-Water Emulsion

By mixing the components given in the table, a cream, the composition of which is also given, was prepared on the basis of a water-in-oil-in-water dispersion.

TABLE 7
W/O/W-Cream

| | |
|---|---|
| Glyceryl stearate | 3.00 |
| PEG-100 stearate | 0.75 |
| Behenyl alcohol | 2.00 |
| Caprylic/capric | 8.0 |
| triglyceride | |
| Octyl dodecanol | 5.00 |
| C12-15 alkyl benzoate | 3.00 |
| Elastase antisense | 0.10 |
| oligonucleotide (SEQ ID NO | |
| 22) | |
| Magnesium sulfate (MgSO4) | 0.80 |
| Ethylenediaminetetraacetic | 0.10 |
| acid | |
| Preservative | q.s |
| Perfume | q.s |
| Water | ad. 100.0 |
| pH value set to 6.0 | |

Analogous creams were obtained by using the hyaluronidase antisense oligonucleotides (SEQ ID NO 28) and (SEQ ID NO 35) or 0.1 wt.-% of a mixture of equal parts of anti-SEQ ID NO 22, anti-SEQ ID NO 28 and anti-SEQ ID NO 35.

That which is claimed:

1. A pharmaceutical or cosmetic composition for topical application, comprising an oligonucleotide or a physiologically compatible salt thereof.

2. The composition according to claim 1, wherein said oligonucleotide or said physiologically compatible salt thereof is capable of hybridizing with an mRNA or a gene sequence which codes for a connective tissue-decomposing enzyme.

3. The composition according to claim 2, wherein the connective tissue-decomposing enzyme includes one or more enzymes selected from the group consisting of collagen-decomposing endopeptidases, elastin-decomposing endopeptidases and hyaluronane-decomposing endo-beta-N-acetylglycosamimidases.

4. The composition according to claim 3, wherein the connective tissue-decomposing enzyme includes one or more collagen-decomposing endopeptidases selected from the group consisting of matrix metalloproteinase 1, 8 and 13.

5. The composition according to claim 4, wherein said oligonucleotide or said physiologically compatible salt thereof is capable of hybridizing with one or more of the sequences SEQ ID NO 1 to SEQ ID NO 13.

6. The composition according to claim 5, wherein said oligonucleotide or said physiologically compatible salt thereof is capable of hybridizing with one or more of the region downstream from nucleotide 1951 onwards, the region from nucleotide 72 to 1481, the translation initiating region (region from nucleotide 72 to 360), the region upstream from nucleotide 71 onwards and the region adjacent to the start sequence (region from nucleotide 72 to 128) of SEQ ID NO 1.

7. The composition according to claim 3, wherein the connective tissue-decomposing enzyme includes an elastin-decomposing endopeptidase and said elastin-decomposing endopeptidase is elastase 2.

8. The composition according to claim 7, wherein said oligonucleotide or said physiologically compatible salt thereof is capable of hybridizing with one or more of the sequences SEQ ID NO 14 to SEQ ID NO 23.

9. The composition according to claim 8, wherein said oligonucleotide or said physiologically compatible salt thereof is capable of hybridizing with one or more of the region downstream from nucleotide 839 onwards (3′ untranslated region), the region from nucleotide 39 to 842 (open reading frame), the translation initiating region (region from nucleotide 39 to 119), the region upstream from nucleotide 39 onwards (5′ untranslated region) and the region adjacent to the start sequence (region from nucleotide 39 to 75) of SEQ ID NO 14.

10. The composition according to claim 3, wherein the connective tissue-decomposing enzyme includes one or more hyaluronane-decomposing endo-beta-N-acetylglycosamimidases selected from the group consisting of hyaluronidase 2 (HYAL2; AK016575), SPAM1 (s67798), HYAL3 (AF036035), HYAL4 (AF009010) and HYAL5 (AF036144).

11. The composition according to claim 10, wherein said oligonucleotide or said physiologically compatible salt thereof is capable of hybridizing with one or more of sequences SEQ ID NO 24 to SEQ ID NO 36.

12. The composition according to claim 11, wherein said oligonucleotide or said physiologically compatible salt thereof is capable of hybridizing with one or more of the region from nucleotide 308 to 1792 (open reading frame), the translation initiating region (region from nucleotide 308 to 498), the region upstream from nucleotide 308 onwards (5′ untranslated region) and the region adjacent to the start sequence (region from nucleotide 308 to 421) of SEQ ID NO 24.

13. The composition according to claim 1, wherein said oligonucleotide or said physiologically compatible salt thereof comprises two or more oligonucleotides or physiologically compatible salts thereof that are capable of hybridizing with (1) the gene sequences or mRNAs of two or more different collagen-decomposing enzymes, elastases or hyaluronidases or (2) two or more different sequence regions of one and the same gene or one and the same mRNA of a collagen-decomposing enzyme, an elastase or a hyaluronidase.

14. The composition according to claim 1, wherein said oligonucleotide or said physiologically compatible salt thereof has a length of 7 to 50 nucleotides.

15. The composition according to claim 1, wherein said oligonucleotide or said physiologically compatible salt thereof has one or more phosphate groups that have been replaced by phosphothioate, methylphosphonate or phosphoramidate groups.

16. The composition according to claim 1, wherein said oligonucleotide or said physiologically compatible salt thereof includes one or more ribose radicals or deoxyribose radicals that have been replaced by amino acid radicals or morpholine radicals.

17. The composition according to claim 1, wherein said oligonucleotide or said physiologically compatible salt thereof includes one or more ribose radicals or deoxyribose radicals that have been modified by fluorine, alkyl or O-alkyl radicals.

18. The composition according to claim 1, wherein said oligonucleotide or said physiologically compatible salt thereof comprises one or more alpha-nucleosides.

19. The composition according to claim 1, comprising from 0.00001 to 10 wt.-% of said oligonucleotide, based on the total weight of the composition.

20. The composition according to claim 1, in the form of a solution, cream, ointment, lotion, hydrodispersion, lipodispersion, emulsion, Pickering emulsion, a gel, a stick or as an aerosol.

21. A method for the care of the skin or for the cosmetic or therapeutic treatment of degenerative skin conditions, comprising the step of applying to the skin a pharmaceutical or cosmetic composition comprising an oligonucleotide or a physiologically compatible salt thereof, said oligonucleotide or said physiologically compatible salt thereof being capable of hybridizing with an mRNA or a gene sequence which codes for a connective tissue-decomposing enzyme.

22. The method according to claim 21, for the treatment of skin changes or skin damage which are caused by UV radiation in the connective tissue, dryness, roughness and slackness of the skin, wrinkling, reduced rehydration by sebaceous glands, and an increased susceptibility to mechanical stress (tendency to crack).

23. The method according to claim 21, for the treatment of photodermatoses, the symptoms of senile xerosis, photoaging and degenerative phenomena which are associated with a decomposition of the connective tissue of the skin.
